(19) United States
(12) Patent Application Publication
(10) Pub. No.: US 2009/0005358 A1
(43) Pub. Date: Jan. 1, 2009

(54) Compositions and methods for treating medical conditions

(76) Inventor: Jan Lessem, Newtown, PA (US)

Correspondence Address: CLARK & ELBING LLP, 101 FEDERAL STREET, BOSTON, MA, 02110, US

(21) Appl. No.: 12/215,005

(22) Filed: Jun. 24, 2008

Related U.S. Application Data
(60) Provisional application No. 60/937,282, filed on Jun. 26, 2007.

(51) Int. Cl. A61K 31/519; A61P 29/00; A61K 31/5377; A61K 31/553; A61K 31/55

(52) U.S. Cl. 514/211.13; 514/262.1; 514/234.2; 514/217

(57) ABSTRACT

The invention features methods, compositions, and kits for the treatment of pain or pruritus in a patient. In one embodiment, the methods, compositions, and kits of the invention provide for a combination therapy including a tricyclic compound and a tetra-substituted pyrimidopyrimidine.

### CROSS-REFERENCE TO RELATED APPLICATIONS

This application claims benefit from U.S. Application No. 60,937,282 filed Jun. 26, 2007, which is incorporated herein by reference.

### BACKGROUND OF THE INVENTION

The invention relates to the treatment of pain or pruritus in a patient in need of such treatment. The invention also relates to the treatment of musculoskeletal disorders, or pain, fatigue, tenderness, impairment in mobility, soft tissue swelling, or bony swelling associated therewith.

Neuropathic, inflammatory, and nociceptive pain differ in their etiology, pathophysiology, diagnosis, and treatment. Nociceptive pain occurs in response to the activation of a specific subset of peripheral sensory neurons, the nociceptors by intense or noxious stimuli. It is generally acute, self-limiting and serves a protective biological function by acting as a warning of potential or on-going tissue damage. It is typically well-localized. Examples of nociceptive pain include but are not limited to traumatic or surgical pain, labor pain, sprains, bone fractures, burns, bumps, bruises, injections, dental procedures, skin biopsies, and obstructions.

Inflammatory pain is pain that occurs in the presence of tissue damage or inflammation including postoperative, post-traumatic pain, arthritic (rheumatoid or osteoarthritis) pain and pain associated with damage to joints, muscle, and tendons as in axial low back pain.

Neuropathic pain is a common type of chronic, non-malignant pain, which is the result of an injury or malfunction in the peripheral or central nervous system and serves no protective biological function. It is estimated to affect more than 1.6 million people in the U.S. population. Neuropathic pain has many different etiologies, and may occur, for example, due to trauma, surgery, herniation of an intervertebral disk, spinal cord injury, diabetes, infection with herpes zoster (shingles), HIV/AIDS, late-stage cancer, amputation (including mastectomy), carpal tunnel syndrome, chronic alcohol use, exposure to radiation, and as an unintended side-effect of neurotoxic treatment agents, such as certain anti-HIV and chemotherapeutic drugs.

In contrast to nociceptive pain, neuropathic pain is frequently described as “burning,” “electric,” “tingling,” or “shooting” in nature. It is often characterized by chronic allodynia (defined as pain resulting from a stimulus that does not ordinarily elicit a painful response, such as light touch) and hyperalgesia (defined as an increased sensitivity to a normally painful stimulus), and may persist for months or years beyond the apparent healing of any damaged tissues.

Pain may occur in patients with cancer, which may be due to multiple causes; inflammation, compression, invasion, metastatic spread into bone or other tissues.

There are some conditions where pain occurs in the absence of a noxious stimulus, tissue damage or a lesion to the nervous system, called dysfunctional pain and these include but are not limited to fibromyalgia, tension type headache, irritable bowel disorders.

Migraine is a headache associated with the activation of sensory fibers innervating the meninges of the brain.

Itch (pruritus) is a dermatological condition that may be localized and generalized and can be associated with skin lesions (rash, atopic eczema, wheals). Itch accompanies many conditions including but not limited to stress, anxiety, UV radiation from the sun, metabolic and endocrine disorders (e.g., liver or kidney disease, hyperthyroidism), cancers (e.g., lymphoma), reactions to drugs or food, parasitic and fungal infections, allergic reactions, diseases of the blood (e.g., polycythemia vera), and dermatological conditions. Itch is mediated by a subset of small diameter primary sensory neurons, the pruriceptor, that share many features of nociceptor neurons, including expression of TRPV1 channels.

Despite the development of a variety of therapies for pain and pruritis, there is a need for additional agents.

### SUMMARY OF THE INVENTION

The invention features methods, compositions, and kits for treating pain or pruritis by administering a tricyclic compound and a tetra-substituted pyrimidopyrimidine or an adenosine activity upregulator to a patient in need of such treatment. The invention also features methods, compositions, and kits for treating a musculoskeletal disorder, or pain, fatigue, tenderness, impairment in mobility, soft tissue swelling, or bony swelling associated with a musculoskeletal disorder, in a patient by administering a tricyclic compound and a tetra-substituted pyrimidopyrimidine or an adenosine activity upregulator to the patient in need thereof.

Accordingly, in a first aspect, the invention features a method for reducing pain in a patient in need thereof, by administering to the patient a tricyclic compound and a tetra-substituted pyrimidopyrimidine or an adenosine activity upregulator, wherein the tricyclic compound and the tetra-substituted pyrimidopyrimidine or adenosine activity upregulator are administered in amounts and for a duration that together are sufficient to reduce pain in the patient. In certain embodiments of the invention, the pain that is reduced is inflammatory pain, neuropathic pain, or nociceptive pain. In related embodiments, the nociceptive pain is caused be surgery, labor, sprains, bone fractures, burns, bumps, bruises, injections, dental procedures, biopsies, or obstructions. In other embodiments, the inflammatory pain that is reduced in a patient is postoperative pain, post-traumatic pain, arthritic pain, or pain associated with damage to joints, muscle, and tendons. In other embodiments, the neuropathic pain that is reduced in a patient is caused trauma, surgery, herniation of an intervertebral disk, spinal cord injury, shingles, HIV/AIDS, late-stage cancer, amputation, and carpal tunnel syndrome. In still other embodiments, the pain that is reduced is dysfunctional pain caused by fibromyalgia, tension type headache, irritable bowel disorders, or migraine.

The invention also features a method for treating pruritus in a patient in need thereof by administering to the patient a tricyclic compound and a tetra-substituted pyrimidopyrimidine or an adenosine activity upregulator, wherein the tricyclic compound and the tetra-substituted pyrimidopyrimidine or adenosine activity upregulator are administered in amounts and for a duration that are together sufficient to treat the patient. In certain embodiments, the pruritus to be treated is caused by rash, atopic eczema, wheals, stress, anxiety, UV radiation from the sun, metabolic and endocrine disorders, cancers, infection, or allergic reaction.

The invention further features a method for treating a musculoskeletal disorder by administering to a patient diagnosed with or at risk of developing a musculoskeletal disorder a tricyclic compound and a tetra-substituted pyrimidopyrimidine or an adenosine activity upregulator, wherein the tricyclic compound and the tetra-substituted pyrimidopyrimidine or adenosine activity upregulator are administered in amounts and for a duration that are together sufficient to treat the patient.

The invention also features a method for treating pain, fatigue, tenderness, impairment in mobility, soft tissue swelling, or bony swelling associated with a musculoskeletal disorder, e.g., osteoarthritis, by administering to a patient diagnosed with or at risk of developing such pain, fatigue, tenderness, impairment in mobility, soft tissue swelling, or bony swelling a tetra-substituted pyrimidopyrimidine or an adenosine activity upregulator, wherein the tricyclic compound and the tetra-substituted pyrimidopyrimidine or adenosine activity upregulator are administered in amounts and for a duration that are together sufficient to treat the patient.

For any of the above methods, the tricyclic compound and the tetra-substituted pyrimidopyrimidine or adenosine activity upregulator can be administered substantially simultaneously (i.e., within an hour of each other) or within 14 days of each other (e.g., within 2, 4, 6, 8, 12, or 16 hours or 1, 5, 7, 10, or 14 days of each other). If desired, the tricyclic compound and the tetra-substituted pyrimidopyrimidine or adenosine activity upregulator can be formulated in a single composition (e.g., for topical or systemic administration) or as separate dosage forms. In one embodiment, the tricyclic compound is amoxapine and the tetra-substituted pyrimidopyrimidine is dipyridamole. In one particular example, 50-100 mg of amoxapine and 200-400 mg of dipyridamole are administered daily. In another example, 10-50 mg of desipramine and 200-400 mg of dipyridamole are administered daily.

In certain embodiments, the tricyclic compound and the tetra-substituted pyrimidopyrimidine are the only two active ingredients (although excipients will generally also be present).

Additional agents can also be administered. Exemplary therapeutic agents are antibiotics, disease-modifying anti-rheumatic drugs (DMARDs), corticosteroids, non-steroidal anti-inflammatory drugs (NSAIDs), anti-convulsants, muscle relaxants, analgesics, cannibinoids, and sedatives. These additional therapeutic agents may be administered within 14 days, 7 days, 1 day, or 12 hours of administration of a tricyclic compound and/or a tetra-substituted pyrimidopyrimidine, or substantially simultaneously therewith. The additional therapeutic agents may be present in the same or different pharmaceutical compositions as the tricyclic compound and/or tetra-substituted pyrimidopyrimidine of the invention. When present in different pharmaceutical compositions, different routes of administration may be used.

The invention further features a kit containing a tricyclic compound, a tetra-substituted pyrimidopyrimidine or adenosine activity upregulator, and instructions for administering the tricyclic compound and the tetra-substituted pyrimidopyrimidine or adenosine activity upregulator to a patient for the treatment of pain.

The invention further feathers a kit containing a tricyclic compound and instructions for administering the tricyclic compound with a tetra-substituted pyrimidopyrimidine or adenosine activity upregulator to a patient for the treatment of pain. In a related embodiment, the invention features a kit containing a tetra-substituted pyrimidopyrimidine or adenosine activity upregulator and instructions for administering the tetra-substituted pyrimidopyrimidine or adenosine activity upregulator with a tricyclic compound to a patient for the treatment of pain.

The invention also features a kit that includes a composition containing a tricyclic compound and a tetra-substituted pyrimidopyrimidine or adenosine activity upregulator, and instructions for administering the composition to a patient for the treatment of pain.

In all the above kits, the pain can be inflammatory pain, neuropathic pain, or nociceptive pain. In particular embodiments of the above kits, the nociceptive pain to be treated is caused by surgery, labor, sprains, bone fractures, burns, bumps, bruises, injections, dental procedures, biopsies, or obstructions, the inflammatory pain to be treated is postoperative pain, post-traumatic pain, arthritic pain, or pain associated with damage to joints, muscle, or tendons, and the neuropathic pain to be treated is caused by trauma, surgery, herniation of an intervertebral disk, spinal cord injury, shingles, HIV/AIDS, late-stage cancer, amputation, or carpal tunnel syndrome. In other embodiments, the pain to be treated is pain caused by fibromyalgia, tension type headache, irritable bowel disorders, or migraine.

The invention further provides a kit containing a tricyclic compound, a tetra-substituted pyrimidopyrimidine or adenosine activity upregulator, and instructions for administering the tricyclic compound and the tetra-substituted pyrimidopyrimidine or adenosine activity upregulator to a patient for the treatment of pruritus.

In a related embodiment, the invention provides a kit containing a tricyclic compound and instructions for administering the tricyclic compound and a tetra-substituted pyrimidopyrimidine or adenosine activity upregulator to a patient for the treatment of pruritus. In another embodiment, the invention features a kit containing a tetra-substituted pyrimidopyrimidine or adenosine activity upregulator and instructions for administering the tetra-substituted pyrimidopyrimidine or adenosine activity upregulator with a tricyclic compound to a patient for the treatment of pruritus.

The invention also provides a kit that includes a composition containing a tricyclic compound and a tetra-substituted pyrimidopyrimidine or adenosine activity upregulator and instructions for administering the composition to a patient for the treatment of pruritus.

In the above kits, the pruritus to be treated can be caused by rash, atopic eczema, wheals, stress, anxiety, UV radiation from the sun, metabolic and endocrine disorders, cancers, infection, or allergic reaction.

In any of the above kits, instructions for administering to the patient a third agent selected from the group of antibiotics, disease-modifying anti-rheumatic drugs, corticosteroids, NSAIDs, anti-convulsants, muscle relaxants, analgesics, cannibinoids, or sedatives are provided.

In certain embodiments of any of the foregoing aspects of the invention, the tricyclic compound is selected from the group consisting of amitriptyline, amoxapine, clomipramine, dothiepin, doxepin, desipramine, imipramine, lofepramine, loxapine, maprotiline, mianserin, mirtazapine, oxaprotiline, nortriptyline, octriptyline, protriptyline, and trimipramine, and the tetra-substituted pyrimidopyrimidine is dipyridamole. In one particular embodiment, the tricyclic compound is amoxapine and the tetra-substituted pyrimidopyrimidine is dipyridamole.

By “tricyclic compound” is meant a compound having one the formulas (I), (II), (III), or (IV):

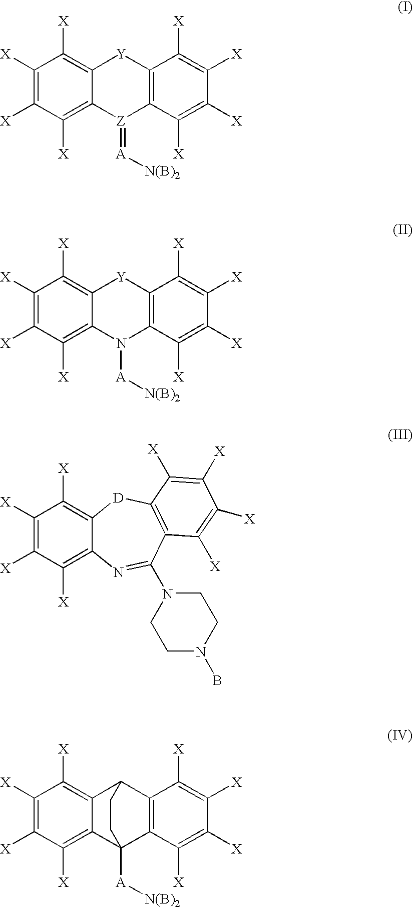

wherein each X is, independently, H, Cl, F, Br, I, CH₃, CF₃, OH, OCH₃, CH₂CH₃, or OCH₂CH₃; Y is CH₂, O, NH, S(O)₀₋₂, (CH₂)₃, (CH)₂, CH₂O, CH₂NH, CHN, or CH₂S; Z is C or S; A is a branched or unbranched, saturated or monounsaturated hydrocarbon chain having between 3 and 6 carbons, inclusive; each B is, independently, H, Cl, F, Br, I, CX₃, CH₂CH₃, OCX₃, or OCX₂CX₃; and D is CH₂, O, NH, or S(O)₀₋₂. In preferred embodiments, each X is, independently, H, Cl, or F; Y is (CH₂)₂, Z is C; A is (CH₂)₃; and each B is, independently, H, Cl, or F. Other tricyclic compounds are described below. Tricyclic compounds include tricyclic antidepressants such as amoxapine, 8-hydroxyamoxapine, 7-hydroxyamoxapine, loxapine (e.g., loxapine succinate, loxapine hydrochloride), 8-hydroxyloxapine, amitriptyline, clomipramine, doxepin, imipramine, trimipramine, desipramine, nortriptyline, and protriptyline, although compounds need not have antidepressant activities to be considered tricyclic compounds of the invention.

By “tetra-substituted pyrimidopyrimidine” is meant a compound of formula (V):

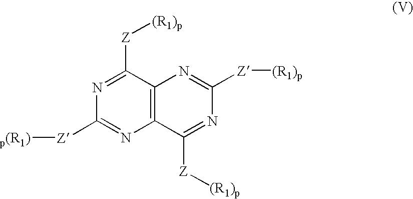

wherein each Z and each Z′ is, independently, N, O, C,

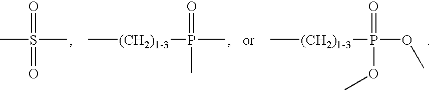

When Z or Z′ is O or

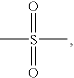

then p=1, when Z or Z′ is N,

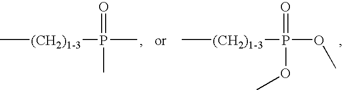

then p=2, and when Z or Z′ is C, then p=3. In formula (V), each R₁ is, independently, X, OH, N-alkyl (wherein the alkyl group has 1 to 20, more preferably 1-5, carbon atoms); a branched or unbranched alkyl group having 1 to 20, more preferably 1-5, carbon atoms; or a heterocycle, preferably as defined in formula (Y), below. Alternatively, when p>1, two R₁ groups from a common Z or Z′ atom, in combination with each other, may represent —(CY₂)ₖ— in which k is an integer between 4 and 6, inclusive. Each X is, independently, Y, CY₃, C(CY₃)₃, CY₂CY₃, (CY₂)₁₋₅OY, substituted or unsubstituted cycloalkane of the structure CₙY₂ₙ₋₁, wherein n=3-7, inclusive. Each Y is, independently, H, F, Cl, Br, or I. In one embodiment, each Z is the same moiety, each Z′ is the same moiety, and Z and Z′ are different moieties.

Particularly useful tetra-substituted pyrimidopyrimidines for use in the methods, kits, and compositions of the invention are dipyridamole (also known as 2,6-bis(diethanolamino)-4,8-dipiperidinopyrimido(5,4-d)pyrimidine); 2,6-disubstituted 4,8-dibenzylaminopyrimido[5,4-d]pyrimidines; mopidamole; dipyridamole monoacetate; R-E 244 (1-((2,7-bis(2-methyl-4-morpholinyl)-6-phenyl-4-pteridinyl)(2-hydroxyethyl)amino)-2-propanol); TX-3301 (asasantin); NU3026 (2,6-di-(2,2-dimethyl-1,3-dioxolan-4-yl)-methoxy-4,8-di-piperidinopyrimidopyrimidine); NU3059 (2,6-bis-(2,3-dimethyoxypropoxy)-4,8-di-piperidinopyrimidopyrimidine); NU3060 (2,6-bis[N,N-di(2-methoxy)ethyl]-4,6-di-piperidinopyrimidopyrimidine); and NU3076 (2,6-bis(diethanolamino)-4,8-di-4-methoxybenzylaminopyrimidopyrimidine). Other tetra-substituted pyrimidopyrimidines are described in U.S. Pat. Nos. 3,031,450 and 4,963,541, hereby incorporated by reference.

By “adenosine activity upregulator” is meant adenosine and any compounds that mimic or potentiate the physiological effects of adenosine, such as adenosine receptor agonists, adenosine transport inhibitors, adenosine kinase inhibitors, and phosphodiesterase (PDE) inhibitors, as described herein.

By “non-steroidal anti-inflammatory drug” or “NSAID” is meant a non-steroidal agent that prevents or diminishes inflammation. NSAIDs include naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, tolmetin, and COX-2 inhibitors such as rofecoxib, celecoxib, valdecoxib, or lumiracoxib.

By “non-steroidal immunophilin-dependent immunosuppressant” or “NsIDI” is meant any non-steroidal agent that decreases proinflammatory cytokine production or secretion, binds an immunophilin, or causes a downregulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

The term “pain” is used herein in the broadest sense and refers to all types of pain, including acute and chronic pain, such as nociceptive pain, e.g. somatic pain and visceral pain; inflammatory pain, dysfunctional pain, neuropathic pain, e.g., centrally generated pain and peripherally generated pain, migraine, and cancer pain.

The term “nociceptive pain” is used to include all pain caused by noxious stimuli that threaten to or actually injure body tissues, including, without limitation, by a cut, bruise, bone fracture, crush injury, burn, and the like. Pain receptors for tissue injury (nociceptors) are located mostly in the skin or in the internal organs.

The term “somatic pain” is used to refer to pain arising from bone, joint, muscle, skin, or connective tissue. This type of pain is typically well localized.

The term “visceral pain” is used herein to refer to pain arising from visceral organs, such as the respiratory, gastrointestinal tract and pancreas, the urinary tract and reproductive organs. Visceral pain includes pain caused by tumor involvement of the organ capsule. Another type of visceral pain, which is typically caused by obstruction of hollow viscus, is characterized by intermittent cramping and poorly localized pain. Visceral pain may be associated with inflammation as in cystitis or reflux esophagitis.

The term “inflammatory pain” includes pain associates with active inflammation that may be caused by trauma, surgery, infection and autoimmune diseases.

The term “neuropathic pain” is used herein to refer to pain originating from abnormal processing of sensory input by the peripheral or central nervous system consequent on a lesion to these systems.

By “musculoskeletal disorder” is meant an immune system-related disorder of the muscles, ligaments, bones, joints, cartilage, or other connective tissue. Among the most commonly-occurring musculoskeletal disorders are various forms of arthritis, e.g., osteoarthritis, rheumatoid arthritis, juvenile rheumatoid arthritis, and gout. Other musculoskeletal disorders include acquired hyperostosis syndrome, acromegaly, ankylosing spondylitis, Behcet's disease, bone diseases, bursitis, cartilage diseases, chronic fatigue syndrome, compartment syndromes, congenital hypothyroidism, congenital myopathies, dentigerous cyst, dermatomyositis, diffuse idiopathic skeletal hyperostosis, Dupuytren's contracture, eosinophilia-myalgia syndrome, fasciitis, Felty's syndrome, fibromyalgia, hallux valgus, infectious arthritis, joint diseases, Kabuki make-up syndrome, Legg-Perthes disease, lupus, Lyme disease, Melas syndrome, metabolic bone diseases, mitochondrial myopathies, mixed connective tissue disease, muscular diseases, muscular dystrophies, musculoskeletal abnormalities, musculoskeletal diseases, myositis, myositis ossificans, necrotizing fasciitis, neurogenic arthropathy, osteitis deformans, osteochondritis, osteomalacia, osteomyelitis, osteonecrosis, osteoporosis, Paget's disease, Pierre Robin syndrome, polymyalgia rheumatica, polymyositis, postpoliomyelitis syndrome, pseudogout, psoriatic arthritis, reactive arthritis, Reiter disease, relapsing polychondritis, renal osteodystrophy, rhabdomyolysis, rheumatic diseases, rheumatic fever, scleroderma, Sever's disease (calceneal apophysitis), Sjögren's syndrome, spinal diseases, spinal stenosis, Still's disease, synovitis, temporomandibular joint disorders, tendinopathy, tennis elbow, tenosynovitis, Tietze's syndrome, and Wegener's granulomatosis.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds. In one embodiment of the invention, the patient subject to a treatment described herein does not have clinical depression, an anxiety or panic disorder, an obsessive/compulsive disorder, alcoholism, an eating disorder, an attention-deficit disorder, a borderline personality disorder, a sleep disorder, a headache, premenstrual syndrome, an irregular heartbeat, schizophrenia, Tourette's syndrome, or phobias.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, ascorbate, aspartate, benzoate, citrate, digluconate, fumarate, glucoheptonate, glycerophosphate, hemisulfate, heptonate, hexanoate, hydrobromide, hydrochloride, hydroiodide, lactate, malate, maleate, malonate, mesylate, oxalate, phosphate, succinate, sulfate, tartrate, thiocyanate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

By “sustained release” or “controlled release” is meant that the therapeutically active component is released from the formulation at a controlled rate such that therapeutically beneficial blood levels (but below toxic levels) of the component are maintained over an extended period of time ranging from e.g., about 12 to about 24 hours, thus, providing, for example, a 12 hour or a 24 hour dosage form.

By “systemic administration” is meant all nondermal routes of administration, and specifically excludes topical and transdermal routes of administration.

As used herein, the term “treating” refers to administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease to improve or stabilize the subject's condition. Thus, in the claims and embodiments, treating is the administration to a subject either for therapeutic or prophylactic purposes.

By “an amount sufficient” is meant the amount of a compound, in a combination of the invention, required to treat or prevent a disease or condition in a clinically relevant manner. A sufficient amount of an active compound used to practice the present invention for therapeutic treatment of particular diseases and conditions caused varies depending upon the manner of administration, the age, body weight, and general health of the patient. Ultimately, the prescribers will decide the appropriate amount and dosage regimen.

Compounds useful in the invention include those described herein in any of their pharmaceutically acceptable salts or other forms, including isomers such as diastereomers and enantiomers, esters, solvates, and polymorphs thereof, as well as racemic mixtures and pure isomers of the compounds described herein. As an example, by “loxapine” is meant the free base, as well as any pharmaceutically acceptable salt thereof (e.g., loxapine hydrochloride, loxapine succinate).

Compounds useful in the invention may also be isotopically labeled. Useful isotopes include hydrogen, carbon, nitrogen, oxygen, phosphorous, fluorine, and chlorine, (e.g., ²H, ³H, ¹³C, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

Other features and advantages of the invention will be apparent from the following detailed description, the drawings, and the claims.

### DETAILED DESCRIPTION

The invention provides methods, compositions, and kits for the treatment of pain or pruritis in a patient in need thereof. When co-administered, includes a tricyclic compound and a tetra-substituted pyrimidopyrimidine or an adenosine activity upregulator can act synergistically to treat pain or pruritis, and thereby allow lower dosages of one or both therapeutic agents to be administered, relative to the dosages required when the tricyclic compound and the tetra-substituted pyrimidopyrimidine or an adenosine activity upregulator are administered alone.

The invention is described in greater detail below.

### Tetra-Substituted Pyrimidopyrimidines

Tetra-substituted pyrimidopyrimidines that are useful in the methods, compositions, and kits of this invention include 2,6-disubstituted 4,8-dibenzylaminopyrimido[5,4-d]pyrimidines. Particularly useful tetra-substituted pyrimidopyrimidines include dipyridamole (also known as 2,6-bis(diethanolamino)-4,8-dipiperidinopyrimido(5,4-d)pyrimidine); mopidamole; dipyridamole monoacetate; R-E 244 (1-((2,7-bis(2-methyl-4-morpholinyl)-6-phenyl-4-pteridinyl)(2-hydroxyethyl)amino)-2-propanol); TX-3301 (asasantin); NU3026 (2,6-di-(2,2-dimethyl-1,3-dioxolan-4-yl)-methoxy-4,8-di-piperidinopyrimidopyrimidine); NU3059 (2,6-bis-(2,3-dimethyoxypropoxy)-4,8-di-piperidinopyrimidopyrimidine); NU3060 (2,6-bis[N,N-di(2-methoxy)ethyl]-4,6-di-piperidinopyrimidopyrimidine); and NU3076 (2,6-bis(diethanolamino)-4,8-di-4-methoxybenzylaminopyrimidopyrimidine). Other tetra-substituted pyrimidopyrimidines are described in U.S. Pat. Nos. 3,031,450 and 4,963,541.

### Adenosine and Adenosine Activity Upregulators

Dipyridamole is an adenosine activity upregulator. If desired, another adenosine activity upregulator can be used in place of dipyridamole in the methods, compositions, and kits of the invention. Suitable adenosine activity upregulators are adenosine receptor agonists, adenosine transport inhibitors, adenosine kinase inhibitors, and phosphodiesterase (PDE) inhibitors, discussed below.

Adenosine Receptor Agonists

Examples of adenosine receptor agonists that can be employed in the methods, compositions, and kits of the invention are adenosine hemisulfate salt, adenosine amine congener solid, N⁶-(4-amino-3-iodophenyl)methyl-5′-N-methylcarboxamidoadenosine (I-AB-MECA); N-((2-methylphenyl)methyl)adenosine (Metrifudil); 2-(1-hexynyl)-N-methyladenosine (HEMADO); N-(1-methyl-2-phenylethyl)adenosine (R-PIA); N⁶-(R-4-hydroxyphenylisopropyl)adenosine (HPIA); N⁶-cyclopentyladenosine (CPA); N⁶-cyclopentyl-2-(3-phenylaminocarbonyltriazene-1-yl)adenosine (TCPA); N-((1S,trans)-2-hydroxycyclopentyl)adenosine (GR 79236); N⁶-cyclohexyladenosine (CHA); 2-chloro-N⁶-cyclopentyladenosine (CCPA); N-ethylcarboxamidoadenosine (NECA); 2-(4-(2-carboxyethyl)phenethylamino)-5′-N-ethylcarboxamidoadenosine (CGS 21680); N⁶-(3-iodobenzyl)-5′-N-methylcarboxamidoadenosine (IB-MECA); 2-(cyclohexylmethylidene hydrazino)adenosine (WRC 0470); 2-(4-(2-carboxyethyl)phenethylamino)-5′-N-ethylcarboxamidoadenosine (CGS 21680); N⁶-(2-(3,5-dimethoxyphenyl)-2-(2-methylphenyl)ethyl)adenosine (DPMA); hexynyladenosine-5′-N-ethylcarboxamide (HE-NECA); 2-[(2-aminoethyl-aminocarbonylethyl)phenylethylamino]-5′-N-ethyl-carboxamidoadenosine (APEC); 2-chloro-N⁶-(3-iodobenzyl)-5′-N-methylcarboxamidoadenosine (2-Cl-IB-MECA); 2-phenylaminoadenosine (CV 1808); 3′-Aminoadenosine-5′-uronamides; CV Therapeutics™ small molecule drugs Tecadenoson (CVT-510); Regadenoson (CVT 3146); and Carisa (CVT 3033); and Aderis Pharmaceuticals™ small drug molecules 2-[2-(4-chlorophenyl)ethoxy]adenosine (MRE 0094), 1-deoxy-1-[6-[[(iodophenyl)methyl]amino]-9H-purine-9-yl]-N-methyl-(-D-ribofuranuronamide) (CF101), Selodenoson (DTI-0009) and Binodenoson (MRE-0470). Other adenosine receptor agonists are those described or claimed in Gao et al., JPET, 298: 209-218 (2001); U.S. Pat. Nos. 5,278,150, 5,877,180, 6,232,297; U.S. Patent Application Publication No. 20050261236, and PCT Publication No. WO/9808855, incorporated herein by reference.

Adenosine Transport Inhibitors

Adenosine transport inhibitors that can be employed in the methods, compositions, and kits of the invention include 3-[1-(6,7-diethoxy-2-morpholinoquinazolin-4-yl)piperidin-4-yl]-1,6-dimethyl-2,4(1H,3H)-quinazolinedione hydrochloride (KF24345); 6-(4-nitrobenzyl)-thioinosine (NBI) and 6-(2-hydroxy-5-nitrobenzyl)-thioguanosine (NBG); 6-[4-(1-cyclohexyl-1H-tetrazol-5-yl)butoxy]-3,4-dihydro-2(1H)-quinolinone (Cilostazol); (2-amino-4,5-dimethyl-3-thienyl)-[3-(trifluoromethyl)phenyl]methanone (PD 81723); 3,7-dihydro-3-methyl-1-(5-oxohexyl)-7-propyl-1H-purine-2,6-dione (propentofylline); 6-[(4-nitrobenzyl)thio]-9-β-D-ribofuranosylpurine (nitrobenzylthioinosine) (NBMR); 3,4,5-trimethoxy-, (tetrahydro-1H-1,4-diazepine-1,4(5H)-diyl)di-3,1-propanediyl benzoic acid, ester (dilazep); hexobendine; dipyridamole; and adenosine transport inhibitors described in Fredholm, J. Neurochem. 62:563-573 (1994), Noji et al., J. Pharmacol. Exp. Ther. 300:200-205 (2002); and Crawley et al.; Neurosci. Lett. 36:169-174 (1983), each of which is incorporated herein by reference.

Adenosine Kinase Inhibitors

Adenosine kinase inhibitors are adenosine activity upregulators that can be used in the methods, compositions, and kits of the invention. Adenosine kinase inhibitors are generally described as either nucleoside-like, or nonnucleoside-like.

Nucleoside-Like Adenosine Kinase Inhibitors

Nucleoside-like adenosine kinase inhibitors that can be used in the methods, compositions, and kits of the invention include 5-iodotubercidin (5IT) and 2-diaryltubercidin analogues; 5′-deoxo-5′-deoxy-5-iodotubercidin (5′d-5IT); and 5′-deoxo-5′-aminoadenosine (NH₂dADO). Other nucleoside-like adenosine kinase inhibitors are described in McGaraughty et al., Current Topics in Medicinal Chemistry 5:43-58 (2005); Ugarkar, J. Med. Chem. 43:2883-2893 (2000); Ugarkar et al., J. Med. Chem. 43:2894-2905 (2000); Kaplan and Coyle, Eur. J. Pharmacol. 1:1-8 (1998); and Sinclair et al. Br. J. Pharmacol. 5:1037-1044 (2001), each of which is incorporated herein by reference.

Nonnucleoside-Like Adenosine Kinase Inhibitors

Nonnucleoside-like adenosine kinase inhibitors that can be used in the methods, compositions, and kits of the invention include 5-bromopyrrolopyrrolidine; 4-amino-5-(3-bromophenyl)-7-(6-morpholino-pyridin-3-yl)pyrido[2,3-d]pyrimidine (ABT-702). Other nonnucleoside-like AK inhibitors are described in McGaraughty et al., Current Topics in Medicinal Chemistry 5:43-58 (2005), Gomtsyan and Lee, Current Pharmaceutical Design 10:1093-1103 (2004); Jarvis et al. J. Pharm. Exp. Ther. 295:1156-1164 (2000); Kowaluk, et al. J. Pharm. Exp. Ther. 295:1165-1174 (2000); and German Patent Application DE 10141212 A1, each of which is incorporated herein by reference.

Phosphodiesterase Inhibitors

Several isozymes of phosphodiesterases act as regulatory switches by catalyzing the degradation of cAMP to adenosine-5-monophosphate (5′-AMP). Inhibitors of phosphodiesterases can lead to an increase in cAMP levels, which in turn can lead to an increase in antiinflammatory actions.

Type I Phosphodiesterase Inhibitors

Type I PDE inhibitors that can be employed in the methods, compositions, and kits of the invention include (3-alpha, 16-alpha)-eburnamenine-14-carboxylic acid ethyl ester (Vinpocetine); 18-methoxymethyl-3-isobutyl-1-methylxantine (MIMX); 1-carboxy-2,3,4,4a,4b,5,6,6a,6b,7,8,8a,8b,9,10,10a,14,16,17,17a,17b,18,19,19a,19b, 20,21,21a,21b,22,23,23a-dotriacontahydro-14-hydroxy-8a,10a-bis(hydroxymethyl)-14-(3-methoxy-3-oxopropyl)-1,4,4a,6,6a,17b,19b,21b-octamethyl beta-D-glucopyranosiduronic acid (Ks-505a); cis-5,6a,7,8,9,9a-hexahydro-2-(4-(trifluoromethyl)phenylmethyl)-5-methyl-cyclopent (4,5)imidazo[2,1-b]purin-4(3H)-one (SCH 51866); and 2-o-propoxyphenyl-8-azapurine-6-one (Zaprinast). Other Type I PDE inhibitors are described in U.S. Patent Application Nos. 20040259792 and 20050075795, incorporated herein by reference.

Type II Phosphodiesterase Inhibitors

Type II PDE inhibitors that can be employed in the methods, compositions, and kits of the invention include erythro-9-(2-hydroxy-3-nonyl)adenine (EHNA); 2,3,6,7-tetrahydro-9,10-dimethoxy-3-methyl-2-((2,4,6-trimethylphenyl)imino)-4H-pyrimido(6,1-a)isoquinolin-4-one (trequinsin); ND7001 (Neuro3D Pharmaceuticals); and BAY 60-7550 (Alexis Biochemicals). Other Type II PDE inhibitors are described in U.S. Patent Application No. 20030176316, incorporated herein by reference.

Type III Phosphodiesterase Inhibitors

Type III PDE inhibitors that can be employed in the methods, compositions, and kits of the invention include 3-isobutyl-1-methylxanthine (IBMX); 6-dihydro-2-methyl-6-oxo-3,4′-bipyridine)-5-carbonitrile (milrinone); and N-cyclohexyl-4-((1,2-dihydro-2-oxo-6-quinolinyl)oxy)-N-methyl-butanamide (cilostamide). Other Type III PDE inhibitors are described in the following patents and patent applications: EP 0 653 426, EP 0 294 647, EP 0 357 788, EP 0 220 044, EP 0 326 307, EP 0 207 500, EP 0 406 958, EP0 150 937, EP0 075 463, EP0 272 914, and EP0 112 987,U.S. Pat. Nos. 4,963,561; 5,141,931, 6,897,229, and 6,156,753; U.S. Patent Application Nos. 20030158133, 20040097593, 20060030611, and 20060025463; WO 96/15117; DE 2825048; DE 2727481; DE 2847621; DE 3044568; DE 2837161; and DE 3021792, each of which is incorporated herein by reference.

Type IV Phosphodiesterase Inhibitors

Type IV PDE inhibitors that can be employed in the methods, compositions, and kits of the invention include 4-(3-cyclopentyloxy-4-methoxyphenyl)-2-pyrrolidone (rolipram) and 4-(3-butoxy-4-methoxybenzyl)-2-imidazolidinone (Ro20-1724). Other Type IV PDE inhibitors are described in the following patents, patent applications, and references: U.S. Pat. Nos. 3,892,777, 4,193,926, 4,655,074, 4,965,271, 5,096,906, 5,124,455, 5,272,153, 6,569,890, 6,953,853, 6,933,296, 6,919,353, 6,953,810, 6,949,573, 6,909,002, and 6,740,655; U.S. Patent Application Nos. 20030187052, 20030187257, 20030144300, 20030130254, 20030186974, 20030220352, 20030134876, 20040048903, 20040023945, 20040044036, 20040106641, 20040097593, 20040242643, 20040192701, 20040224971, 20040220183, 20040180900, 20040171798, 20040167199, 20040146561, 20040152754, 20040229918, 20050192336, 20050267196, 20050049258, 20060014782, 20060004003, 20060019932, 20050267196, 20050222207, 20050222207, 20060009481; PCT Publication No. WO 92/079778; and Molnar-Kimber, K. L. et al. J. Immunol., 150:295A (1993), each of which is incorporated herein by reference.

Type V Phosphodiesterase Inhibitors

Type V PDE inhibitors that can be used in the methods, compositions, and kits of the invention include those described in U.S. Pat. Nos. 6,992,192, 6,984,641, 6,960,587, 6,943,166, 6,878,711, and 6,869,950, and U.S. Patent Application Nos. 20030144296, 20030171384, 20040029891, 20040038996, 20040186046, 20040259792, 20040087561, 20050054660, 20050042177, 20050245544, 20060009481, each of which is incorporated herein by reference.

Type VI Phosphodiesterase Inhibitors

Type VI PDE inhibitors that can be used in the methods, compositions, and kits of the invention include those described in U.S. Patent Application Nos. 20040259792, 20040248957, 20040242673, and 20040259880, each of which is incorporated herein by reference.

Type VII Phosphodiesterase Inhibitors

Type VII PDE inhibitors that can be used in the methods, compositions, and kits of the invention include those described in the following patents, patent application, and references: U.S. Pat. Nos. 6,838,559, 6,753,340, 6,617,357, and 6,852,720; U.S. Patent Application Nos. 20030186988, 20030162802, 20030191167, 20040214843, and 20060009481; PCT Publication WO 00/68230; and Martinez et al., J. Med. Chem. 43:683-689 (2000), each of which is incorporated herein by reference.

Non-Selective Phosphodiesterase Inhibitors

Non-selective PDE inhibitors that can be used in the methods, compositions, and kits of the invention include theophylline, papaverine, and ibudilast. Other PDE inhibitors that can be used in the methods, compositions, and kits of the invention are described in U.S. Pat. No. 6,953,774.

### Tricyclic Compounds

Tricyclic compounds that can be used in the methods, compositions, and kits of the invention include those encompassed by any one of formulas (I)-(IV), above, and amitriptyline, amoxapine, clomipramine, desipramine, dothiepin, doxepin, imipramine, lofepramine, maprotiline, mianserin, mirtazapine, nortriptyline, octriptyline, oxaprotiline, protriptyline, trimipramine, 10-(4-methylpiperazin-1-yl)pyrido(4,3-b)(1,4)benzothiazepine; 11-(4-methyl-1-piperazinyl)-5H-dibenzo(b,e)(1,4)diazepine; 5,10-dihydro-7-chloro-10-(2-(morpholino)ethyl)-11H-dibenzo(b,e)(1,4)diazepin-11-one; 2-(2-(7-hydroxy-4-dibenzo(b,f)(1,4)thiazepine-11-yl-1-piperazinyl)ethoxy)ethanol; 2-chloro-11-(4-methyl-1-piperazinyl)-5H-dibenzo(b,e)(1,4)diazepine; 4-(1H-dibenz(b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; (Z)-2-butenedioate 5H-dibenzo(b,e)(1,4)diazepine; adinazolam; amineptine; amitriptylinoxide; butriptyline; clothiapine; clozapine; demexiptiline; 11-(4-methyl-1-piperazinyl)-dibenz(b,f)(1,4)oxazepine; 11-(4-methyl-1-piperazinyl)-2-nitro-dibenz(b,f)(1,4)oxazepine; 2-chloro-11-(4-methyl-1-piperazinyl)-dibenz(b,f)(1,4)oxazepine monohydrochloride; dibenzepin; 11-(4-methyl-1-piperazinyl)-dibenzo(b,f)(1,4)thiazepine; dimetacrine; fluacizine; fluperlapine; imipramine N-oxide; iprindole; lofepramine; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; noxiptilin; opipramol; oxaprotiline; perlapine; pizotyline; propizepine; quetiapine; quinupramine; tianeptine; tomoxetine; flupenthixol; clopenthixol; piflutixol; chlorprothixene; and thiothixene. Other tricyclic compounds are described, for example, in U.S. Pat. Nos. 2,554,736; 3,046,283; 3,310,553; 3,177,209; 3,205,264; 3,244,748; 3,271,451; 3,272,826; 3,282,942; 3,299,139; 3,312,689; 3,389,139; 3,399,201; 3,409,640; 3,419,547; 3,438,981; 3,454,554; 3,467,650; 3,505,321; 3,527,766; 3,534,041; 3,539,573; 3,574,852; 3,622,565; 3,637,660; 3,663,696; 3,758,528; 3,922,305; 3,963,778; 3,978,121; 3,981,917; 4,017,542; 4,017,621; 4,020,096; 4,045,560; 4,045,580; 4,048,223; 4,062,848; 4,088,647; 4,128,641; 4,148,919; 4,153,629; 4,224,321; 4,224,344; 4,250,094; 4,284,559; 4,333,935; 4,358,620; 4,548,933; 4,691,040; 4,879,288; 5,238,959; 5,266,570; 5,399,568; 5,464,840; 5,455,246; 5,512,575; 5,550,136; 5,574,173; 5,681,840; 5,688,805; 5,916,889; 6,545,057; and 6,600,065, and phenothiazine compounds that fit Formula (I) of U.S. patent application Ser. Nos. 10/617,424 or 60/504,310.

### Corticosteroids

If desired, the co-administration of tricyclic compound and a tetra-substituted pyrimidopyrimidine or an adenosine activity upregulator may be performed in conjunction with one or more corticosteroids. Corticosteroids that are useful in the methods, compositions, and kits of this invention are selected from the class of selective glucocorticosteroid receptor agonists (SEGRAs) including, without limitation, 11-alpha, 17-alpha,21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21 (beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

### Non-Steroidal Anti-Inflammatory Drugs (NSAIDs)

If desired, the co-administration of tricyclic compound and a tetra-substituted pyrimidopyrimidine or an adenosine activity upregulator may be performed in conjunction one or more non-steroidal anti-inflammatory drugs (NSAIDs), such as naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin. Two or more NSAIDs can be administered in the same treatment.

### Disease-Modifying Anti-Rheumatic Drugs

Disease modifying anti-rheumatic drugs (DMARDs) can be used in the methods, compositions, and kits of the invention. DMARDs are a class of anti-inflammatory drugs. DMARDs known in the art include, but are not limited to anakinra, auranofin, aurothioglucose, azathioprine, chlorambucil, cyclophosphamide, cyclosporine, D-penicillamine, gold sodium thiomalate (injectable gold), hydroxychloroquine, leflunomide, methotrexate, minocycline, mycophenol mofetil, or sulfasalazine. Two or more DMARDs can be also administered in the same treatment.

### Therapy

The invention features methods for treating pain or pruritis in a patient in need thereof. While the examples describe the combination of a single tricyclic compound and a tetra-substituted pyrimidopyrimidine, it is understood that the combination of multiple agents is often desirable. In addition, one more tricyclic compounds and one or more tetra-substituted pyrimidopyrimidines may be co-administered with additional agents (e.g., antibiotics, corticosteroids, DMARDs, or NSAIDs).

Therapy according to the invention may be performed alone or in conjunction with another therapy and may be provided at home, the doctor's office, a clinic, a hospital's outpatient department, or a hospital. The duration of the therapy depends on the type of disease or disorder being treated, the age and condition of the patient, the stage and type of the patient's disease, and how the patient responds to the treatment.

In particular embodiments of any of the methods of the invention, the compounds are administered simultaneously or within fourteen days, ten days, five days, 24 hours, or 1 hour of each other in amounts sufficient to treat the patient. The compounds may be formulated together as a single composition, or may be formulated and administered separately (e.g., separate dosage forms). One or both compounds may be administered in a low dosage or in a high dosage, each of which is defined herein. It may be desirable to administer to the patient other compounds, such as an NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), NsIDIs (e.g., cyclosporine, tacrolimus, pimecrolimus, and ISAtx247), antibiotics, or DMARDs.

In combination therapy of the invention, the dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds.

In the therapy, one agent (e.g., a tricyclic compound) may be administered to a patient in a first treatment period, followed by administration of both the first agent and a second agent (e.g., a tetra-substituted pyrimidopyrimidine) during a second treatment period, followed by a third treatment period, wherein only the first agent is administered alone, wherein the first, second, and third treatment periods are within a continuous treatment regimen.

The compounds in question may be administered orally in the form of tablets, capsules, elixirs or syrups, or rectally in the form of suppositories. Parenteral administration of the compound is suitably performed, for example, in the form of saline solutions or with the compound incorporated into liposomes. In cases where the compound in itself is not sufficiently soluble to be dissolved, a solubilizer such as ethanol can be applied. Additionally, the compositions may be formulated for epidural or intrathecal administration.

Desirably, the methods, compositions, and kits of the invention are more effective than other methods, compositions, and kits. By “more effective” is meant that a method, composition, or kit exhibits greater efficacy, is less toxic, safer, more convenient, better tolerated, or less expensive, or provides more treatment satisfaction than another method, composition, or kit with which it is being compared.

### Cotherapy

If desired, one or more additional agents may be administered in conjunction with the methods, compositions, and kits of the invention. Suitable agents include antibiotics (minocycline, penicillin, cephalosporin, tetracycline, oxytetracycline, chlortetracycline, metronidazole, chloramphenicol, streptomycin, neomycin, sulfonamides, phenolic compounds, quarternary ammonium compounds, doxycycline); antiseptics (e.g., chlorhexidine); nonsteroidal antiinflammatories (e.g., flurbiprofen, carprofen, diclofenac, fenbufen, fenclozic acid, fenoprofen, flufenamic acid, ibuprofen, indomethacin, indoprofen, ketoprofen, lonazolac, loxoprofen, meclofenamic acid, mefanamic acid, naproxen, proprionic acids, salicylic acids, sulindac, tolmetin, meloxicam, oxicams, piroxicam, tenoxicam, etodolac, and oxaprozin); tranexamic acid, allantoin; epsilon-aminocaproic acid; lysozyme; dihydrocholesterol; beta-glycyrrhetinic acid; platelet aggregation inhibitors (e.g., abciximab, aspirin, cilostazol, clopidogrel, eptifibatide, ticlopidine, or tirofiban); anticoagulants (e.g., dalteparin, danaparoid, enoxaparin, heparin, tinzaparin, or warfarin); antipyretics (e.g., acetaminophen); ticlopidine; clopidogrel; angiotensin converting enzyme inhibitors; beta blockers; pentoxifylline; cilostazol; estrogen replacement therapy; and lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These agents may be administered concomitantly or within 14 days of the method of the invention. If desired, one or more of the foregoing agents is coformulated with one or more agents of the invention to form a single composition. Thus, in one embodiment, the invention features a tricyclic compound, one of the foregoing agents, and a tetra-substituted pyrimidopyrimidine or an adenosine activity upregulator.

Osteoarthritis

The methods, compositions, and kits of the invention may be used for the treatment of pain associated with osteoarthritis. If desired, one or more agents typically used to treat osteoarthritis may be used with the methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), NsIDIs (e.g., cyclosporine, tacrolimus, pimecrolimus, and ISAtx247), or analogs thereof. Thus, in one embodiment, the invention features the combination of an SSRI with any of the foregoing agents and a corticosteroid in the methods and kits for the treatment of osteoarthritis or pain associated therewith.

Rheumatoid Arthritis

The methods, compositions, and kits of the invention may be used for the treatment of pain associated with rheumatoid arthritis. If desired, one or more agents typically used to treat rheumatoid arthritis may be used with the methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., infliximab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine.

Pain

The methods, compositions, and kits of the invention may be used for the treatment of pain (e.g., neuropathic pain or nociceptive pain). If desired, one or more agents typically used to treat pain may be used with the corticosteroid therapy methods, compositions, and kits of the invention. Such agents include NSAIDs, opioids, tricyclic antidepressants, anticonvulsants, amantadine, tramadol, oxycodone, buproprion, mexiletine, capsaicin, muscle relaxants, pregabalin, ketamide, analgesics, SSRIs, cannibinoids, sedatives, and anti-anxiety drugs.

Anticonvulsants

The anticonvulsants are used in prevention of the occurrence of epileptic seizures. The goal of an anticonvulsant is to suppress the rapid and excessive firing of neurons that start a seizure. Many anticonvulsants block sodium (Na+) channels, calcium (Ca2+) channels, AMPA receptors, or NMDA receptors. Some anticonvulsants inhibit the metabolism of GABA or increase its release.

Anti-convulsants include barbiturates (e.g., amobarbital, aprobarbital, barbital, butabarbital, butalbital, hexobarbital, methohexital, pentobarbital, secobarbital, sodium thiopental, talbutal, thiobarbital, Phenobarbital, methylphenobarbital, metharbital, barbexaclone), benzodiazepines (e.g., alprazolam, bromazepam, chlordiazepoxide, cinolazepam, clonazepam, clorazepate, diazepam, estazolam, flunitrazepam, flurazepam, halazepam, ketazolam, loprazolam, lorazepam, lormetazepam, medazepam, midazolam, nitrazepam, nordazepam, oxazepam, phenazepam, pinazepam, prazepam, quazepam, temazepam, tetrazepam, and triazolam), carboxamide (e.g., carbamazepine and oxcarbazepine), vigabatrin, progabide, and tiagabine topiramate, gabapentin, pregabalin, hydantoins (e.g., ethotoin, phenyloin, mephenyloin, and fosphenyloin), oxazolidinediones (e.g., paramethadione, trimethadione, ethadione), beclamide, primidone, pyrrolidines (e.g., brivaracetam, levetiracetam, and seletracetam), succinimides (e.g., ethosuximide, phensuximide, and mesuximide), sulfonamides (e.g., acetazolamide, sulthiame, methazolamide, and zonisamide), lamotrigine, pheneturide, phenacemide, valpromide, valnoctamide, and valproate.

Muscle Relaxants

A muscle relaxant is a drug that decreases the tone of a muscle. Muscle relaxants include methocarbamol, baclofen, carisoprodol, chlorzoxazone, cyclobenzaprine, dantrolene, metaxalone, orphenadrine, pancuronium, tizanidine, and dicyclomine.

Analgesics

Analgesics are compounds used to treat pain. Analgesics include opioids (e.g., morphine, codeine, thebaine, oxycodone, hydrocodone, dihydrocodeine, hydromorphone, oxymorphone, nicomorphine, methadone, levo-alphacetylmethadol, fentanyl, alfentanil, sufentanil, remifentanil, ketobemidone, carfentanyl, ohmefentanyl, ketobemidone, allylprodine, prodine, PEPAP, propoxyphene, dextropropoxyphene, dextromoramide, bezitramide, piritramide, pentazocine, phenazocine, buprenorphine, butorphanol, nalbufine, levorphanol, levomethorphan, dezocine, etorphine, lefetamine, tilidine, tramadol, naloxone, and naltrexone), NSAIDs (e.g., naproxen sbdium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), acetaminophen, and COX-2 inhibitors (e.g., rofecoxib, celecoxib, valdecoxib, and lumiracoxib).

Cannibinoids

Cannabinoids are a group of diterpene C21 compounds present in Cannabis sativa L and include a group of substances that are structurally related to THC or that bind to cannabinoid receptors. Cannibinoids include CP-55940, HU-210, SR141716, SR144528, WIN 55, 212-2, JWH-133, Nabilone, Levonantradol, Marinol, and Sativex.

Sedatives

A sedative is a substance that depresses the central nervous system (CNS), resulting in calmness, relaxation, reduction of anxiety, sleepiness, slowed breathing, slurred speech, staggering gait, poor judgment, and slow, uncertain reflexes. Sedatives include chlorpromazine, fluphenazine, haloperidol, loxapine succinate, perphenazine, prochlorperazine, thiothixene, trifluoperazine, clozapine, olanzapine, quetiapine, risperidone, ziprasidone, catnip, Kava Kava, Mandrake, valerian, chloral hydrate, diethyl ether, eszopiclone, ethchlorvynol, ethyl alcohol, gamma-hydroxybutyrate, glutethimide, meprobamate, methaqualone, methyl trichloride, methyprylon, ramelteon, zaleplon, zolpidem, and zopiclone.

Thus, in one embodiment, the invention features the any of the foregoing agents in combination with a tricyclic compound and a tetra-substituted pyrimidopyrimidine for treating pain.

The pain treated using the methods, compositions, and kits of the invention include pain caused as a result of neuropathy, including diabetic neuropathy, polyneuropathy, cancer pain, fibromyalgia, myofascial pain syndrome, osteoarthritis, pancreatic pain, pelvic/perineal pain, post herpetic neuralgia, rheumatoid arthritis, sciatica/lumbar radiculopathy, spinal stenosis, temporo-mandibular joint disorder, HIV pain, trigeminal neuralgia, chronic neuropathic pain, lower back pain, failed back surgery pain, back pain, post-operative pain, post physical trauma pain (including gunshot, road traffic accident, burns), cardiac pain, chest pain, pelvic pain/PID, joint pain (tendonitis, bursitis, acute arthritis), neck pain, bowel pain, phantom limb pain, obstetric pain (labor/C-Section), renal colic, acute herpes zoster pain, acute pancreatitis breakthrough pain (cancer), and dysmenorhoea/endometriosis. The methods, compositions, and kits of the invention can also be used to treat pain caused as a result of inflammatory disease, or as a result of combined inflammatory, autoimmune and neuropathic tissue damage, including rheumatoid arthritis, osteoarthritis, rheumatoid spondylitis, gouty arthritis, and other arthritic conditions, cancer, HIV, chronic pulmonary inflammatory disease, silicosis, pulmonary sarcosis, bone resorption diseases, reperfusion injury (including damage caused to organs as a consequence of reperfusion following ischaemic episodes e.g. myocardial infarcts, strokes), autoimmune damage (including multiple sclerosis, Guillam Barre Syndrome, myasthenia gravis) graft v. host rejection, allograft rejections, fever and myalgia due to infection, AIDS related complex (ARC), keloid formation, scar tissue formation, Crohn's disease, ulcerative colitis and pyresis, irritable bowel syndrome, osteoporosis, cerebral malaria and bacterial meningitis, bowel pain, cancer pain, back pain, fibromyalgia, and post-operative pain.

### Formulation of Pharmaceutical Compositions

The administration of a combination of the invention may be by any suitable means that results in treatment in a patient in need thereof. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The compositions may be formulated according to conventional pharmaceutical practice (see, e.g., Remington: The Science and Practice of Pharmacy, 20th edition, 2000, ed. A.R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time period after administration, using controlled release formulations.

Administration of compounds in controlled release formulations is useful where the compound, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD50) to median effective dose (ED50)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the therapeutic compound. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Each compound of the combination may be formulated in a variety of ways that are known in the art. For example, the first and second agents may be formulated together or separately. Desirably, the first and second agents are formulated together for the simultaneous or near simultaneous administration of the agents.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions.

The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

The two compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Topical Formulations

Compositions can also be adapted for topical use with a topical vehicle containing from between 0.0001% and 25% (w/w) or more of the tricyclic compound and between 0.001% and 25% (w/w) or more of the tetra-substituted pyrimidopyrimidine.

Inhalation

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

Dosages

The dosage of each compound employed in the methods, compositions, and kits of the invention depends on several factors, including: the administration method, the condition to be treated, the severity of the condition, whether the condition is to be treated or prevented, and the age, weight, and health of the person to be treated. Amoxapine can be administered one to four times daily in an amount as low as 10, 25, 50, 60, 75, or 100 mg and has high as 100, 200, 250, 300, or 500 mg. In one embodiment, amoxapine is orally administered once per day in an amount between 50 and 100 mg/dose. Desipramine can be administered one to four times daily in an amount as low as 1, 5, 7.5, 10, 20, or 50 mg and has high as 50, 100, 150, 200, or 250 mg. In one embodiment, desipramine is orally administered once or twice per day in an amount between 10 and 50 mg/dose. Dipyridamole can be administered one to four times daily in an amount as low as 50, 75, 100, 150, or 200 mg and has high as 200, 250, 300, 400, or 500 mg. In one embodiment, dipyridamole is orally administered two times per day in an amount between 100 and 200 mg/dose.

Administration of each drug in the combination can, independently, be one to four times daily for one day to one year, and may even be for the life of the patient. Chronic, long-term administration will be indicated in many cases.

As described above, each compound may be administered orally in the form of tablets, capsules, elixirs or syrups, or rectally in the form of suppositories, such that the drugs are absorbed into the bloodstream. Parenteral administration of a compound is suitably performed, for example, in the form of saline solutions or with the compound incorporated into liposomes. In cases where the compound in itself is not sufficiently soluble to be dissolved, a solubilizer such as ethanol can be applied.

### Pain, Function, and Fatigue Indices

In order to measure the efficacy of any of the methods, compositions, or kits of the invention, a measurement index may be used. Indices that are useful in the methods, compositions, and kits of the invention include a visual analog scale (VAS), a Likert scale, the Lequesne index, the WOMAC index, the AUSCAN index, the Piper Fatigue Scale, and the Multidimensional Assessment of Fatigue (MAF) scale, each of which is well known in the art. Such indices may be used to measure pain, function, fatigue, stiffness, tenderness, impairment in mobility, soft tissue swelling, bony swelling, or other variables.

A visual analog scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain by choosing the spot on the line that best corresponds to the sensation of pain, where one end of the line corresponds to “no pain” (score of 0 cm) and the other end of the line corresponds to “unbearable pain” (score of 10 cm). This procedure provides a simple and rapid approach to obtaining quantitative information about how the patient is experiencing pain. VAS scales can also be used, e.g., to measure fatigue. VAS scales and their use are described, e.g., in U.S. Pat. Nos. 6,709,406 and 6,432,937.

A Likert scale similarly provides a measure of a one-dimensional quantity. Generally, a Likert scale has discrete integer values ranging from a low value (e.g., 0, meaning no pain) to a high value (e.g., 7, meaning extreme pain). A patient experiencing pain is asked to choose a number between the low value and the high value to represent the degree of pain experienced. Likert scales can also be used, e.g., to measure fatigue. Likert scales and their use are described, e.g., in U.S. Pat. Nos. 6,623,040 and 6,766,319.

The Lequesne index and the Western Ontario and McMaster Universities (WOMAC) osteoarthritis index assess pain, function, and stiffness in the knee and hip of OA patients using self-administered questionnaires. Both knee and hip are encompassed by the WOMAC, whereas there is one Lequesne questionnaire for the knee and a separate one for the hip. These questionnaires are useful because they contain more information content in comparison with VAS or Likert. Both the WOMAC index and the Lequesne index questionnaires have been extensively validated in OA, including in surgical settings (e.g., knee and hip arthroplasty). Their metric characteristics do not differ significantly.

The AUSCAN (Australian-Canadian hand arthritis) index employs a valid, reliable, and responsive patient self-reported questionnaire. In one instance, this questionnaire contains 15 questions within three dimensions (Pain, 5 questions; Stiffness, 1 question; and Physical function, 9 questions). An AUSCAN index may utilize, e.g., a Likert or a VAS scale.

The Piper Fatigue scale is a 41-item measure of fatigue developed for research purposes and tested with oncology patients (Piper et al. (1989), The development of an instrument to measure the subjective dimension of fatigue. In S. Funk, E. Tornquist, M. Champagne, & R. Wiese (Eds.). Key aspects of comfort: Management of pain, fatigue, and nausea (pp. 199-207). New York: Springer.) The Multidimensional Assessment of Fatigue (MAF) scale, a revision of the Piper Fatigue scale, contains 15 items and measures four dimensions of fatigue: severity (#1-2), distress (#3), degree of interference in activities of daily living (#4-14), and frequency (#15), with scores ranging from 1 (no fatigue) to 50 (severe fatigue). The MAF has been validated in RA patients (Belza, J. Rheumatol. 22:639-643, 1995).

### Rheumatoid Arthritis Indices

In order to measure the efficacy of any of the methods, compositions, or kits of the invention, a measurement index may be used. Indices that are useful in the methods, compositions, and kits of the invention include the ACR-20/50/70 and the disease activity score (DAS).

ACR-20/50/70

ACR-20/50/70 is a widely accepted composite index of improvement in RA proposed by the American College of Rheumatology (ACR). ACR-20/50/70 refers to a composite improvement of 20%, 50% or 70% in swollen joint count, tender joint count, and 3 or more of the following 5 measures: patient's own global assessment of RA disease activity; physician's global assessment of disease activity; patient's own assessment of pain due to RA; acute-phase reactant (CRP); and patient's self-addressed disability (Health Assessment Questionnaire).

DAS28

The disease activity score (DAS) is a combined index that was developed in Nijmegen in the 1980s to measure the disease activity in patients with RA. It has been extensively validated for its use in clinical trials in combination with the European League Against Rheumatism (EULAR) response criteria. To calculate the DAS28, the number of swollen joints and tender joints should be assessed using 28-joint counts, the CRP levels should be measured in mg/L, and the patients general health (GH) or global disease activity measured on a Visual Analog Scale (VAS) of 100 mm must be obtained. Using this data, the DAS28 using CRP (mg/L) can be calculated using the following formula:

DAS28=0.56*sqrt(tender28)+0.28*sqrt(swollen28)+0.36*ln(CRP+1)+0.014*GH+0.96

The DAS28 provides a number between 0 and 10 indicating the current activity of RA in the patient. A DAS above 5.1 means high disease activity, and below 3.2 indicates low activity. Remission is achieved by a DAS28 lower than 2.6.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLES

### Study Protocol

We conducted a six week blinded, randomized study including daily oral treatment with amoxapine and dipyridamole plus DMARD therapy, with regular CRP and inflammatory cytokine measurements. The study population had active rheumatoid arthritis. The subject must otherwise have been in good general health.

During the study, subjects attended the following study visits:
Screening Visit (Visit 1)
Day 1 (Baseline Visit/Visit 2)
Day 14±1 day (Visit 3)
Day 21±2 days (Visit 4)
Day 42±1 day (Visit 5)

All eligible subjects received DMARD therapy in a standard dose. Subjects were evaluated for study eligibility at the Screening visit, which was conducted within 14 days before the first dose of study drug. The subject provided written informed consent to participate in the study before any Screening laboratory samples were collected or evaluations performed.

Subjects were randomized into treatment groups and received either amoxapine and dipyridamole or placebo tablets. In the treatment group there was one dose escalation after day 14 as follows.
Days 1-14 Dose Level 1 (50 mg amoxapine and 200 mg dipyridamole)
Days 15-42 Dose Level 2 (100 mg amoxapine and 200 mg dipyridamole)
   The drugs were blister packed as follows:

| | | |
|---|---|---|
| 8 am | 8 am | 1 pm |

Treatment group

| | | | |
|---|---|---|---|
| Days 1-14 | 100 mg | 50 mg amoxapine | 100 mg |
| | dipyridamole | | dipyridamole |
| Days 15-42 | 100 mg | 100 mg amoxapine | 100 mg |
| | dipyridamole | | dipyridamole |

Placebo group

| | | | |
|---|---|---|---|
| Days 1-42 | placebo | placebo | placebo |

The results of the study are shown in Tables 1-4. As is shown in Table 1, there was a statistically significant difference (p<0.0482 one tailed Wilcoxon Rank Sum test) in reduction of the distribution of percent change from baseline to Day 42 in DAS28. The treatment group had a median reduction of 8.94% while the placebo patients only reduced by 0.81%. As is shown in Table 2, there was also a statistically significant difference (p<0.0057 one tailed Wilcoxon Rank Sum test) in reduction of the distribution of percent change from baseline to Day 42 in tender joint counts. The treatment group had a median reduction of 30.28% while the placebo patients showed a median % change of 0.00. As is shown in Table 3, there was a statistically significant difference (p<0.0258 one tailed Wilcoxon Rank Sum test) in reduction of the distribution of percent change from baseline to Day 42 in pain, with the treatment group exhibiting a median reduction of 24.57%, compared to 2.64% in the placebo patients. Finally, there was a statistically significant difference (p<0.0238 one tailed Wilcoxon Rank Sum test) in reduction of the distribution of percent change from baseline to Day 42 in patients global assessment of the past week. The treatment group had a median reduction of 16.01% while the placebo patients showed an increase of 0.81%.

TABLE 1
Summary of DAS28
ITT Population (LOCF)

| | | Placebo + DMARD | | | DP/Amox + DMARD | |
|---|---|---|---|---|---|---|
| | | (N = 20) | | | (N = 44) | |
| | | Change from | % Change from | | Change from | % Change from |
| Visit | Value | baseline | baseline | Value | baseline | baseline |

Baseline

| | | |
|---|---|---|
| N | 20 | 44 |
| Mean (Std Dev) | 5.88 (1.090) | 5.44 (0.914) |
| Median | 6.05 | 5.34 |
| Min, Max | 3.9, 7.9 | 3.5, 7.5 |

Day 7

| | | | | | | |
|---|---|---|---|---|---|---|
| N | 1 | 1 | 1 | 4 | 4 | 4 |
| Mean (Std Dev) | 6.04 (.) | −0.27 (.) | −4.26 (.) | 5.54 (0.895) | −0.05 (0.381) | 0.39 (8.121) |
| Median, | 6.04 | −0.27 | −4.26 | 5.51 | −0.16 | −2.77 |
| Min, Max | 6.0, 6.0 | −0.3, −0.3 | −4.3, −4.3 | 4.6, 6.6 | −0.4, 0.5 | −5.3, 12.4 |

Day 14

| | | | | | | |
|---|---|---|---|---|---|---|
| N | 20 | 20 | 20 | 44 | 44 | 44 |
| Mean (Std Dev) | 5.46 (1.436) | −0.42 (1.222) | −6.50 (19.272) | 5.03 (1.020) | −0.41 (0.913) | −6.80 (16.294) |
| Median | 5.61 | 0.00 | 0.00 | 4.99 | −0.25 | −4.96 |
| Min, Max | 2.6, 7.8 | −3.9, 0.8 | −58.0, 13.5 | 2.9, 7.2 | −3.1, 1.2 | −41.0, 27.5 |
| p-value | | | | | 0.2701 | 0.2944 |

Day 21

| | | | | | | |
|---|---|---|---|---|---|---|
| N | 20 | 20 | 20 | 44 | 44 | 44 |
| Mean (Std Dev) | 5.66 (1.504) | −0.22 (1.357) | −2.54 (22.154) | 5.00 (0.984) | −0.44 (0.992) | −6.61 (20.085) |
| Median | 5.74 | 0.05 | 0.96 | 4.97 | −0.50 | −8.67 |
| Min, Max | 2.4, 8.1 | −4.1, 1.4 | −62.0, 24.2 | 3.0, 7.2 | −2.5, 2.2 | −41.7, 61.7 |
| p-value | | | | | 0.0202 | 0.0223 |

Day 28

| | | | | | | |
|---|---|---|---|---|---|---|
| N | 1 | 1 | 1 | 5 | 5 | 5 |
| Mean (Std Dev) | 7.08 (.) | 0.77 (.) | 12.24 (.) | 5.42 (1.089) | −0.16 (0.716) | −2.02 (14.183) |
| Median | 7.08 | 0.77 | 12.24 | 5.18 | −0.14 | −2.48 |
| Min, Max | 7.1, 7.1 | 0.8, 0.8 | 12.2, 12.2 | 4.4, 7.2 | −1.0, 0.7 | −15.7, 18.4 |

Day 42/EOS

| | | | | | | |
|---|---|---|---|---|---|---|
| N | 20 | 20 | 20 | 44 | 44 | 44 |
| Mean (Std Dev) | 5.61 (1.430) | −0.27 (1.353) | −3.13 (22.114) | 4.86 (1.045) | −0.58 (1.126) | −9.20 (20.878) |
| Median | 5.84 | −0.05 | −0.81 | 4.88 | −0.51 | −8.94 |
| Min, Max | 2.1, 8.1 | −4.6, 1.4 | −68.7, 27.6 | 1.8, 6.9 | −3.5, 1.5 | −65.8, 30.8 |
| p-value | | | | | 0.0526 | 0.0482 |

Note:
p-value derived from a Wilcoxon Rank sum test which tests if the center of the distribution of change (% changes) is significantly lower in one treatment group than another.
Note:
Data from Day 7 and Day 28 carried forward, but LOCF not used from previous visit.

TABLE 2
Summary of Tender Joint Counts (VAS)
ITT Population (LOCF)

| | | Placebo + DMARD | | | DP/Amox + DMARD | |
|---|---|---|---|---|---|---|
| | | (N = 20) | | | (N = 44) | |
| | | Change from | % Change from | | Change from | % Change from |
| Visit | Value | baseline | baseline | Value | baseline | baseline |

Baseline

| | | |
|---|---|---|
| N | 20 | 44 |
| Mean (Std Dev) | 22.3 (11.78) | 18.4 (9.89) |
| Median | 23.0 | 17.0 |
| Min, Max | 7, 53 | 5, 47 |

Day 7

| | | | | | | |
|---|---|---|---|---|---|---|
| N | 1 | 1 | 1 | 4 | 4 | 4 |
| Mean (Std Dev) | 23.0 (.) | −3.0 (.) | −11.54 | 18.5 (7.05) | 1.8 (2.99) | 11.79 (15.584) |
| Median | 23.0 | −3.0 | −11.54 | 19.0 | 1.0 | 8.85 |
| Min, Max | 23, 23 | −3, −3 | −11.5, −11.5 | 11, 25 | −1, 6 | −3.8, 33.3 |

Day 14

| | | | | | | |
|---|---|---|---|---|---|---|
| N | 20 | 20 | 20 | 44 | 44 | 44 |
| Mean (Std Dev) | 20.2 (11.72) | −2.1 (7.95) | −4.37 (42.128) | 15.0 (10.02) | −3.4 (5.39) | −21.65 (34.235) |
| Median | 18.0 | −0.5 | −1.79 | 14.0 | −3.0 | −16.03 |
| Min, Max | 0, 44 | −20, 13 | −100.0, 88.9 | 0, 40 | −20, 5 | −100.0, 50.0 |
| p-value | | | | | 0.1215 | 0.0484 |

Day 21

| | | | | | | |
|---|---|---|---|---|---|---|
| N | 20 | 20 | 20 | 44 | 44 | 44 |
| Mean (Std Dev) | 21.6 (14.84) | −0.8 (10.61) | 4.72 (45.780) | 15.0 (9.98) | −3.4 (7.43) | −14.90 (68.304) |
| Median | 18.0 | 0.0 | 0.00 | 15.0 | −4.0 | −17.42 |
| Min, Max | 0, 68 | −32, 15 | −100.0, 88.9 | 0, 42 | −20, 21 | −100.0, 350.0 |
| p-value | | | | | 0.0123 | 0.0065 |

Day 28

| | | | | | | |
|---|---|---|---|---|---|---|
| N | 1 | 1 | 1 | 5 | 5 | 5 |
| Mean (Std Dev) | 26.0 (.) | 0.0 (.) | 0.00 | 15.0 (6.40) | −2.6 (2.41) | −15.48 (18.128) |
| Median | 26.0 | 0.0 | 0.00 | 16.0 | −2.0 | −11.11 |
| Min, Max | 26, 26 | 0, 0 | 0.0, 0.0 | 7, 22 | −6, 0 | −46.2, 0.0 |

Day 42/EOS

| | | | | | | |
|---|---|---|---|---|---|---|
| N | 20 | 20 | 20 | 44 | 44 | 44 |
| Mean (Std Dev) | 22.8 (14.19) | 0.5 (10.31) | 16.08 (65.464) | 13.1 (9.70) | −5.3 (10.09) | −22.20 (56.428) |
| Median | 22.5 | 0.0 | 0.00 | 10.5 | −4.0 | −30.28 |
| Min, Max | 0, 68 | −27, 15 | −100.0, 214.3 | 0, 39 | −34, 22 | −100.0, 183.3 |
| p-value | | | | | 0.0084 | 0.0057 |

Note:
p-value derived from a Wilcoxon Rank sum test which tests if the center of the distribution of change (% changes) is significantly lower in one treatment group than another.
Note:
Data from Day 7 and Day 28 carried forward, but LOCF not used from previous visit.

TABLE 3
Summary of Patient Pain (VAS) Assessment
ITT Population (LOCF)

| | | Placebo + DMARD | | | DP/Amox + DMARD | |
|---|---|---|---|---|---|---|
| | | (N = 20) | | | (N = 44) | |
| | | Change from | % Change from | | Change from | % Change from |
| Visit | Value | baseline | baseline | Value | baseline | baseline |

Baseline

| | | |
|---|---|---|
| N | 20 | 44 |
| Mean (Std Dev) | 58.8 (23.85) | 55.2 (19.69) |
| Median | 64.5 | 63.0 |
| Min, Max | 22, 100 | 15, 91 |

Day 7

| | | | | | | |
|---|---|---|---|---|---|---|
| N | 1 | 1 | 1 | 4 | 4 | 4 |
| Mean (Std Dev) | 34.0 (.) | 6.0 (.) | 21.43 (.) | 47.0 (14.45) | 0.5 (25.04) | 15.38 (57.668) |
| Median | 34.0 | 6.0 | 21.43 | 51.0 | 8.5 | 17.33 |
| Min, Max | 34, 34 | 6, 6 | 21.4, 21.4 | 27, 59 | −36, 21 | −57.1, 84.0 |

Day 14

| | | | | | | |
|---|---|---|---|---|---|---|
| N | 20 | 20 | 20 | 44 | 44 | 44 |
| Mean (Std Dev) | 54.2 (23.23) | −4.6 (27.65) | 10.31 (73.719) | 48.3 (19.57) | −7.0 (19.90) | −6.55 (40.631) |
| Median | 60.5 | −6.0 | −7.49 | 51.5 | −3.5 | −8.08 |
| Min, Max | 13, 92 | −63, 52 | −82.9, 236.4 | 10, 83 | −49, 38 | −77.8, 116.7 |
| p-value | | | | | 0.3947 | 0.3274 |

Day 21

| | | | | | | |
|---|---|---|---|---|---|---|
| N | 20 | 20 | 20 | 44 | 44 | 44 |
| Mean (Std Dev) | 51.9 (24.41) | −6.9 (25.28) | −1.45 (44.451) | 46.5 (21.39) | −8.7 (21.46) | −10.30 (46.536) |
| Median | 53.0 | −2.5 | −4.62 | 45.5 | −11.5 | −17.00 |
| Min, Max | 6, 90 | −70, 28 | −92.1, 63.6 | 3, 86 | −47, 52 | −89.7, 152.9 |
| p-value | | | | | 0.1641 | 0.1169 |

Day 28

| | | | | | | |
|---|---|---|---|---|---|---|
| N | 1 | 1 | 1 | 5 | 5 | 5 |
| Mean (Std Dev) | 67.0 (.) | 39.0 (.) | 139.29 (.) | 30.4 (9.84) | −12.6 (10.92) | −27.14 (13.531) |
| Median | 67.0 | 39.0 | 139.29 | 31.0 | −9.0 | −24.00 |
| Min, Max | 67, 67 | 39, 39 | 139.3, 139.3 | 19, 41 | −32, −6 | −50.8, −18.0 |

Day 42/EOS

| | | | | | | |
|---|---|---|---|---|---|---|
| N | 20 | 20 | 20 | 44 | 44 | 44 |
| Mean (Std Dev) | 55.1 (23.18) | −3.7 (24.07) | 5.50 (53.009) | 41.9 (22.55) | −13.4 (24.86) | −18.03 (48.486) |
| Median | 59.5 | −2.0 | −2.64 | 37.0 | −11.5 | −24.57 |
| Min, Max | 12, 89 | −61, 38 | −81.5, 135.7 | 9, 94 | −64, 60 | −86.4, 176.5 |
| p-value | | | | | 0.0374 | 0.0258 |

Note:
p-value derived from a Wilcoxon Rank sum test which tests if the center of the distribution of change (% changes) is significantly lower in one treatment group than another.
Note:
Data from Day 7 and Day 28 carried forward, but LOCF not used from previous visit.

TABLE 4
Summary of Patient Global in past week (VAS)
ITT Population (LOCF)

| | | Placebo + DMARD | | | DP/Amox + DMARD | |
|---|---|---|---|---|---|---|
| | | (N = 20) | | | (N = 44) | |
| | | Change from | % Change from | | | % Change from |
| Visit | Value | baseline | baseline | Value | Change from baseline | baseline |

Baseline

| | | |
|---|---|---|
| N | 20 | 44 |
| Mean (Std Dev) | 60.2 (22.53) | 57.8 (17.57) |
| Median | 61.0 | 57.5 |
| Min, Max | 20, 100 | 11, 88 |

Day 7

| | | | | | | |
|---|---|---|---|---|---|---|
| N | 1 | 1 | 1 | 4 | 4 | 4 |
| Mean (Std Dev) | 60.0 (.) | 9.0 (.) | 17.65 (.) | 42.0 (17.49) | −8.3 (29.00) | −6.68 (48.095) |
| Median | 60.0 | 9.0 | 17.65 | 41.0 | 1.5 | 2.96 |
| Min, Max | 60, 60 | 9, 9 | 17.6, 17.6 | 22, 64 | −49, 13 | −69.0, 36.4 |

Day 14

| | | | | | | |
|---|---|---|---|---|---|---|
| N | 20 | 20 | 20 | 44 | 44 | 44 |
| Mean (Std Dev) | 59.9 (22.51) | −0.3 (22.33) | 11.97 (58.650) | 50.0 (19.17) | −7.8 (20.82) | −8.09 (40.565) |
| Median | 65.5 | −1.0 | −1.39 | 53.5 | −5.0 | −7.22 |
| Min, Max | 18, 93 | −43, 45 | −70.5, 160.7 | 10, 78 | −62, 39 | −77.3, 127.6 |
| p-value | | | | | 0.1555 | 0.1538 |

Day 21

| | | | | | | |
|---|---|---|---|---|---|---|
| N | 20 | 20 | 20 | 44 | 44 | 44 |
| Mean (Std Dev) | 55.3 (25.85) | −4.9 (25.38) | 1.63 (52.061) | 45.2 (21.58) | −12.6 (23.84) | −16.20 (45.029) |
| Median | 50.0 | 0.0 | 0.00 | 47.5 | −8.5 | −14.58 |
| Min, Max | 4, 93 | −81, 30 | −95.3, 150.0 | 6, 82 | −64, 45 | −89.5, 121.6 |
| p-value | | | | | 0.0510 | 0.0510 |

Day 28

| | | | | | | |
|---|---|---|---|---|---|---|
| N | 1 | 1 | 1 | 5 | 5 | 5 |
| Mean (Std Dev) | 70.0 (.) | 19.0 (.) | 37.25 (.) | 37.0 (15.08) | −17.4 (16.68) | −29.50 (22.457) |
| Median | 70.0 | 19.0 | 37.25 | 30.0 | −9.0 | −19.57 |
| Min, Max | 70, 70 | 19, 19 | 37.3, 37.3 | 26, 63 | −45, −4 | −63.4, −11.3 |

Day 42/EOS

| | | | | | | |
|---|---|---|---|---|---|---|
| N | 20 | 20 | 20 | 44 | 44 | 44 |
| Mean (Std Dev) | 56.4 (25.57) | −3.8 (25.62) | 0.75 (45.328) | 43.1 (22.91) | −14.6 (26.95) | −19.05 (48.294) |
| Median | 64.0 | 0.5 | 0.81 | 40.0 | −9.0 | −16.01 |
| Min, Max | 7, 88 | −71, 30 | −88.5, 93.8 | 7, 93 | −65, 56 | −89.2, 151.4 |
| p-value | | | | | 0.0288 | 0.0238 |

Note:
p-value derived from a Wilcoxon Rank sum test which tests if the center of the distribution of change (% changes) is significantlv lower in one treatment group than another.
Note:
Data from Day 7 and Day 28 carried forward, but LOCF not used from previous visit.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for reducing pain in a patient in need thereof, said method comprising administering to said patient (i) a tricyclic compound; and (ii) an tetra-substituted pyrimidopyrimidine, wherein said tricyclic compound and said tetra-substituted pyrimidopyrimidine are administered in amounts and for a duration that together are sufficient to reduce pain in said patient.

2. The method of claim 1, wherein said pain is inflammatory pain, neuropathic pain, or nociceptive pain.

3. The method of claim 2, wherein said pain is nociceptive pain caused by surgery, labor, sprains, bone fractures, burns, bumps, bruises, injections, dental procedures, biopsies, or obstructions.

4. The method of claim 2, wherein said pain is inflammatory pain selected from postoperative pain, post-traumatic pain, arthritic pain, or pain associated with damage to joints, muscle, and tendons.

5. The method of claim 2, wherein said pain is neuropathic pain caused by trauma, surgery, herniation of an intervertebral disk, spinal cord injury, shingles, HIV/AIDS, late-stage cancer, amputation, and carpal tunnel syndrome.

6. The method of claim 1, wherein said pain is dysfunctional pain caused by fibromyalgia, tension type headache, irritable bowel disorders, or migraine.

7. The method of claim 1, wherein said tricyclic compound is amitriptyline, amoxapine, clomipramine, dothiepin, doxepin, desipramine, imipramine, lofepramine, loxapine, maprotiline, mianserin, mirtazapine, oxaprotiline, nortriptyline, octriptyline, protriptyline, and trimipramine.

8. The method of claim 1, wherein said tetra-substituted pyrimidopyrimidine is dipyridamole, 2,6-disubstituted 4,8-dibenzylaminopyrimido[5,4-d]pyrimidines; mopidamole, dipyridamole monoacetate, 1-((2,7-bis(2-methyl-4-morpholinyl)-6-phenyl-4-pteridinyl)(2-hydroxyethyl)amino)-2-propanol, asasantin, 2,6-di-(2,2-dimethyl-1,3-dioxolan-4-yl)-methoxy-4,8-di-piperidinopyrimidopyrimidine, 2,6-bis-(2,3-dimethyoxypropoxy)-4,8-di-piperidinopyrimidopyrimidine, 2,6-bis[N,N-di(2-methoxy)ethyl]-4,6-di-piperidinopyrimidopyrimidine, or 2,6-bis(diethanolamino)-4,8-di-4-methoxybenzylaminopyrimidopyrimidine.

9. The method of claim 1, wherein said tricyclic compound and said tetra-substituted pyrimidopyrimidine are administered simultaneously or within 14 days of each other.

10. The method of claim 1, wherein said tricyclic compound and said tetra-substituted pyrimidopyrimidine are formulated in a single composition.

11. The method of claim 10, wherein said composition is formulated for topical administration.

12. The method of claim 10, wherein said composition is formulated for systemic administration.

13. The method of claim 10, wherein said composition is formulated for oral administration.

14. The method of claim 1, wherein said tricyclic compound is amoxapine or desipramine and said tetra-substituted pyrimidopyrimidine is dipyridamole.

15. The method of claim 14, wherein 100 to 400 mg of dipyridamole is administered to said patient daily.

16. The method of claim 15, wherein 200 to 400 mg of dipyridamole is administered daily.

17. The method of claim 14, wherein 50 to 100 mg of amoxapine is administered to said patient daily.

18. The method of claim 14, wherein 10 to 50 mg of desipramine is administered to said patient daily.

19. The method of claim 14, wherein said amoxapine or desipramine and said dipyridamole are formulated in separate dosage forms.

20. The method of claim 1, further comprising administering to said patient a third agent selected from the group consisting of antibiotics, disease-modifying anti-rheumatic drugs (DMARDs), non-steroidal anti-inflammatory drugs (NSAIDs), anti-convulsants, muscle relaxants, analgesics, cannibinoids, and sedatives.

21. A method for treating pruritus in a patient in need thereof, said method comprising administering to said patient (i) a tricyclic compound; and (ii) an tetra-substituted pyrimidopyrimidine, wherein said tricyclic compound and said tetra-substituted pyrimidopyrimidine are administered in amounts and for a duration that together are sufficient to treat said patient.

22. The method of claim 21, wherein said pruritus is caused by rash, atopic eczema, wheals, stress, anxiety, UV radiation from the sun, metabolic and endocrine disorders, cancers, infection, or allergic reaction.

23. A method for treating pain or fatigue associated with a musculoskeletal disorder, said method comprising administering to a patient diagnosed with or at risk of developing said pain or fatigue (i) a tricyclic compound; and (ii) an tetra-substituted pyrimidopyrimidine, wherein said tricyclic compound and said tetra-substituted pyrimidopyrimidine are administered in amounts and for a duration that together are sufficient to treat said patient.

24. A method for treating tenderness, impairment in mobility, soft tissue swelling, or bony swelling associated with a musculoskeletal disorder, said method comprising administering to a patient diagnosed with or at risk of developing said tenderness, impairment in mobility, soft tissue swelling, or bony swelling (i) a tricyclic compound; and (ii) an tetra-substituted pyrimidopyrimidine, wherein said tricyclic compound and said tetra-substituted pyrimidopyrimidine are administered in amounts and for a duration that together are sufficient to treat said patient.

25. A kit comprising:
(i) a tricyclic compound;
(ii) an tetra-substituted pyrimidopyrimidine; and
(iii) instructions for administering said tricyclic compound and said tetra-substituted pyrimidopyrimidine to a patient for the treatment of pain or pruritis.

26. A kit comprising:
(i) a tricyclic compound; and
(ii) instructions for administering said tricyclic compound with an tetra-substituted pyrimidopyrimidine to a patient for the treatment of pain or pruritis.

27. A kit comprising:
(i) an tetra-substituted pyrimidopyrimidine; and
(ii) instructions for administering said tetra-substituted pyrimidopyrimidine with a tricyclic compound to a patient for the treatment of pain or pruritis.

28. A kit comprising:
(i) a composition comprising a tricyclic compound and an tetra-substituted pyrimidopyrimidine; and
(ii) instructions for administering said composition to a patient for the treatment of pain or pruritis.
